(19) United States
(12) Patent Application Publication
(10) Pub. No.: US 2012/0158100 A1
(43) Pub. Date: Jun. 21, 2012

(54) Driving Microneedle Arrays into Skin and Delivering RF Energy

(76) Inventor: Kevin Schomacker, Maynard, MA (US)

(21) Appl. No.: 13/165,675

(22) Filed: Jun. 21, 2011

Related U.S. Application Data
(60) Provisional application No. 61/356,967, filed on Jun. 21, 2010.

(51) Int. Cl. A61N 1/18

(52) U.S. Cl. 607/101

(57) ABSTRACT

A skin treatment includes applying vacuum to draw skin into contact with a plate defining a plurality of holes, inserting a plurality of needles through the plurality of holes into the skin, and delivering RF energy via the plurality of needles to treat the skin.

### CROSS-REFERENCE TO RELATED APPLICATION

This application claims the benefit of and priority to U.S. Provisional Patent Application No. 61/356,967 filed Jun. 21, 2010, which is owned by the assignee of the instant application and the disclosure of which is incorporated herein by reference in its entirety.

### FIELD OF THE INVENTION

The invention relates generally to an array of needles held retracted in a protective cartridge. The array of needles can be deployed into the skin through a plate with the assistance of vacuum to effect a skin treatment using radio frequency (RF) energy.

### BACKGROUND

U.S. Pat. No. 6,277,116 to Utely et al. discloses the use of microneedle arrays to shrink collagen by driving a multitude of needles into dermis and delivering RF energy. Utely also teaches the use of insulated needles to confine ohmic heating to the distal needle tip. However, this patent fails to address how to overcome the difficulties of preventing skin from being pushed out of the way by the microneedle array, particularly when the spacings between the needles are small (bed-of-nails effect).

U.S. Pat. No. 6,743,211 to Prausnitz et al. discloses means for reducing skin elasticity to improve microneedle array penetration into tissue. In Prausnitz, skin elasticity is reduced via stretching, pulling or pinching the skin and includes vacuum suction as a means for pulling on the skin. However, this patent fails to teach the use of vacuum to press skin against a plate, and combining pressure and reduced temperature to improve patient comfort by reducing pain. It also fails to describe stretching tissue immediately around the point of needle entry.

### SUMMARY OF THE INVENTION

An array of needles can be held retracted in a protective cartridge. The array of needles can be deployed by an electric solenoid or pneumatic driver, which is used to drive the needle array into skin at a desired velocity. The array can be an array of microneedles. The needles can be used to deliver RF energy to the skin. The proximal shafts of the needles can be insulated to confine ohmic heating to the tips of the needles. This can protect against damage to the epidermis and papillary dermis, or the epidermis and dermis when targeting subcutaneous fatty tissues.

Vacuum or suction can be applied to the skin to lift the skin against the needles. The vacuum can compress the skin against a plate, through which the needles can be deployed. The plate can be a cooling plate, which can alleviate or inhibit pain. The plate can be perforated to assist vacuum suction around each needle and allow needle passage. A cooling plate can protect the superficial skin from injury by the RF energy by lowering the temperature of the epidermis, papillary dermis and/or dermis by removing or conducting heat from the tissue.

The needle array can be used to treat a variety of cosmetic and dermatologic conditions. For example, the needle array can be used to shrink collagen in the dermis for cosmetic improvement of rhytides, wrinkles, skin laxity, etc. Fat volume can be reduced by driving the needles deeper into the hypodermis. A skin laxity treatment or skin tightening can be performed following laser lipolysis.

Driving a multitude of needles into the skin can be difficult due to the “bed-of-nails” effect. The combination of vacuum suction and rapid insertion of the needles can overcome this issue. Keeping the insertion depth of all needles constant across all needles is difficult due to unequal displacement of tissue by the needles and uneven surface of tissue. The use of vacuum suction compressing tissue against a flat plate allows for better control of needle insertion depth. Needle insertion and dermal heating can be uncomfortable to the patient.

The use of vacuum suction and/or contact surface cooling can reduce, inhibit or alleviate pain and improve patient comfort.

In one aspect, there is an apparatus for treating skin. The apparatus includes a housing, a plate disposed in the housing, a vacuum port, a needle array, an actuator mechanism and a source of RF energy. The housing defines a volume. The housing includes a ceiling and a wall extending from the ceiling. The wall has an edge for contacting the skin, and the edge of the wall defines an aperture in the housing. The plate intersects the wall and divides the volume of the housing into two sub-volumes so that the wall extends above and below the plate. A first sub-volume is defined by the ceiling, the wall and a top surface of the plate. A second sub-volume is defined by a bottom surface of the plate, the wall and the aperture defined by the edge of the wall. The plate defines a plurality of holes so that the first sub-volume and the second sub-volume are in fluid communication. The vacuum port is defined in a wall or ceiling of the housing. Vacuum is applied to draw the skin though the aperture and into contact with the bottom surface of the plate. The needle array includes a plurality of needles extending from a base. Each needle is aligned with a respective hole defined in the plate. The actuator mechanism is in communication with the needle array. The actuator mechanism is configured to cause the plurality of needles to pass through their respective holes into the skin. A source of RF energy, radiation or current is in electrical communication with the plurality of needles.

In another aspect, there is a method of treating skin including applying vacuum to draw skin into contact with a plate defining a plurality of holes, inserting a plurality of needles through the plurality of holes into the skin, and delivering RF energy via the plurality of needles to treat the skin.

In yet another aspect, there is an apparatus for treating skin including means for applying vacuum to draw skin into contact with a plate defining a plurality of holes, means for inserting a plurality of needles through the plurality of holes into the skin, and means for delivering RF energy via the plurality of needles to treat the skin. The apparatus can include means for cooling the plate.

In certain embodiments, the plate can be disposed in a housing defining a volume. The housing includes a ceiling and a wall extending from the ceiling. The wall has an edge for contacting the skin, and the edge of the wall defines an aperture in the housing. The plate intersects the wall and divides the volume of the housing into two sub-volumes so that the wall extends above and below the plate. A first sub-volume is defined by the ceiling, the wall and a top surface of the plate. A second sub-volume is defined by a bottom surface of the plate, the wall and the aperture defined by the edge of the wall. The first sub-volume and the second sub-volume are in fluid communication via the plurality of holes.

In still another aspect, there is an apparatus for treating skin. The apparatus includes a housing and a plate. The housing defines a volume, and includes a ceiling and a wall extending from the ceiling. The wall has an edge for contacting the skin. The edge of the wall defines an aperture in the housing. The plate is disposed in the housing, intersects the wall and divides the volume of the housing into two sub-volumes so that the wall extends above and below the plate. A first sub-volume is defined by the ceiling, the wall and a top surface of the plate. A second sub-volume is defined by a bottom surface of the plate, the wall and the aperture defined by the edge of the wall. The plate defines a plurality of holes so that the first sub-volume and the second sub-volume are in fluid communication. The housing defines a vacuum port, and the ceiling of the housing defines a passage. The apparatus includes a needle array including a base having a top surface and a bottom surface, and a plurality of needles extending from the bottom surface of the base, and a plunger slidably insertable into the passage. Each needle is aligned with a respective hole defined in the plate, and the plunger is connected to the top surface of the base. The holes aligned with the needles can be made to provide a sliding guide to each of the needles thereby preventing the needles from buckling when the needles are inserted into the skin. This can allow for finer gage needles to be used, which can be less painful to the patient when inserted. A source of vacuum is in fluid communication with the volume of the housing. The vacuum is applied to draw the skin though the aperture in the housing into contact with the plate. An actuator is in communication with the plunger. The actuator is adapted to slide the plunger through the passage to cause the plurality of needles to pass through the holes into the skin. A source of RF radiation is in electrical communication with the plurality of needles.

In other examples, any of the aspects above, or any apparatus, system or device, or method, process or technique, described herein, can include one or more of the following features. Each needle can include (i) a shaft covered with an insulating material and (ii) an exposed tip to deliver the RF energy. The actuator mechanism can be or can include a solenoid. The actuator mechanism can be adapted to drive the plurality of needles into the skin at a velocity of at least 100 mm/s. The velocity can be at least 1 m/s. The velocity can be achieved when the needle tips first contact the surface of the skin.

The housing can define a passage through the ceiling. The actuator mechanism can include a plunger slidably insertable into the passage and connected to a top surface of the base. The actuator mechanism can be configured to slide the plunger through the passage to drive the plurality of needles into the skin. In certain embodiments, the actuator mechanism includes a hammer configured to strike the base of the plurality of needles and drive the plurality of needles into the skin.

In various embodiments, at least one needle can be is hollow. A temperature sensor or other sensor can be inserted into a bore defined in the at least one needle. In some embodiments, a fluid, medication, anesthetic, or other substance can be delivered through the needle(s). In some embodiments, a highly conductive fluid can be delivered to allow the RF current to diffuse from the needles and reduce the heating of the tissue immediately surrounding the needles.

The polarity of all of the needles can be the same, or some needles can have a different polarity than others. For example, a first subset of the plurality of needles has an oscillating polarity and a second subset of the plurality of needles has the opposite polarity (i.e., 180⁰ out of phase with first polarity). Different treatment patterns can be created in the skin by proper selection of polarity and energy.

In certain embodiments, the plate includes or is coupled to a cooling mechanism. The plate can be thermally conductive. Applying vacuum can form a bleb of skin in each hole, into which each needle is inserted.

In certain embodiments, a controller can be used to sequence the insertion of the needles after a controllable time delay after the skin has contacted the plate, and to initiate RF energy delivery after a controllable time after the insertion of the needles. This allows the skin to be cooled to a selected depth before needle insertion and before RF energy delivery to minimize pain and achieve a desired temperature profile in the treated tissue.

Other aspects and advantages of the invention will become apparent from the following detailed description, taken in conjunction with the accompanying drawings, illustrating the principles of the invention by way of example only.

### BRIEF DESCRIPTION OF THE INVENTION

The advantages of the invention described above, together with further advantages, may be better understood by referring to the following description taken in conjunction with the accompanying drawings. The drawings are not necessarily to scale, emphasis instead generally being placed upon illustrating the principles of the invention.

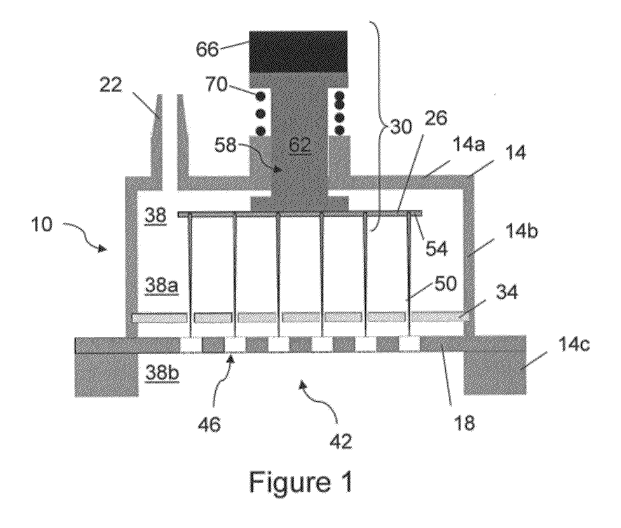

FIG. 1 shows an apparatus for deploying a needle array into skin.

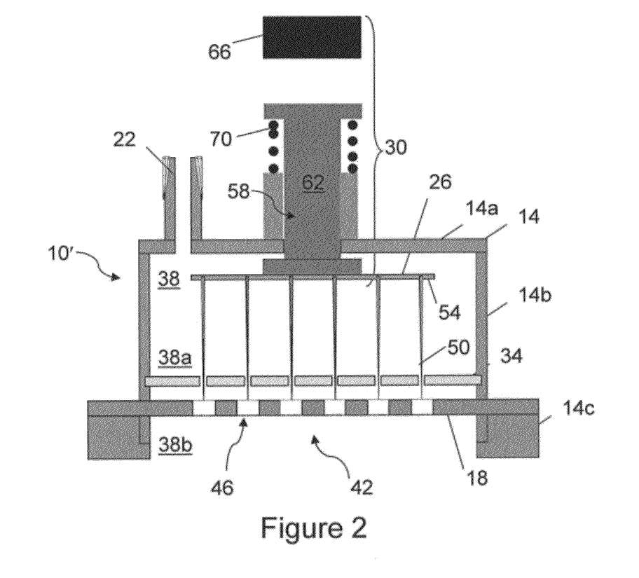

FIG. 2 shows another apparatus for deploying a needle array into skin.

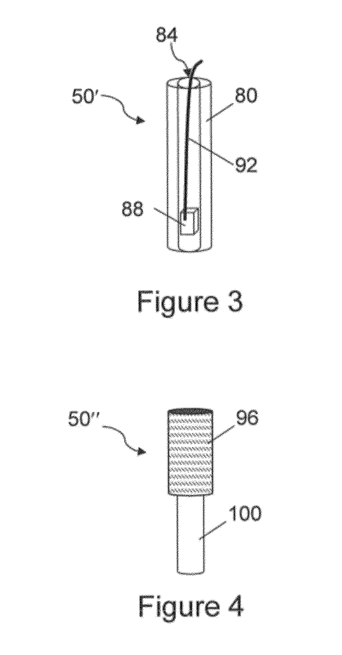

FIG. 3 shows an embodiment of a hollow needle.

FIG. 4 shows a needle having a shaft covered in an insulating material and having an exposed tip to deliver the RF energy.

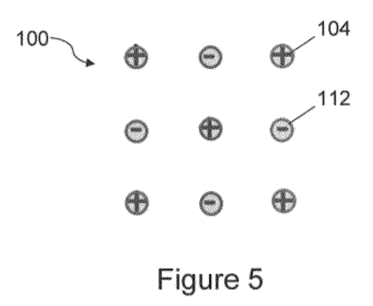

FIG. 5 shows an exemplary damage pattern for biaxial alternating poles.

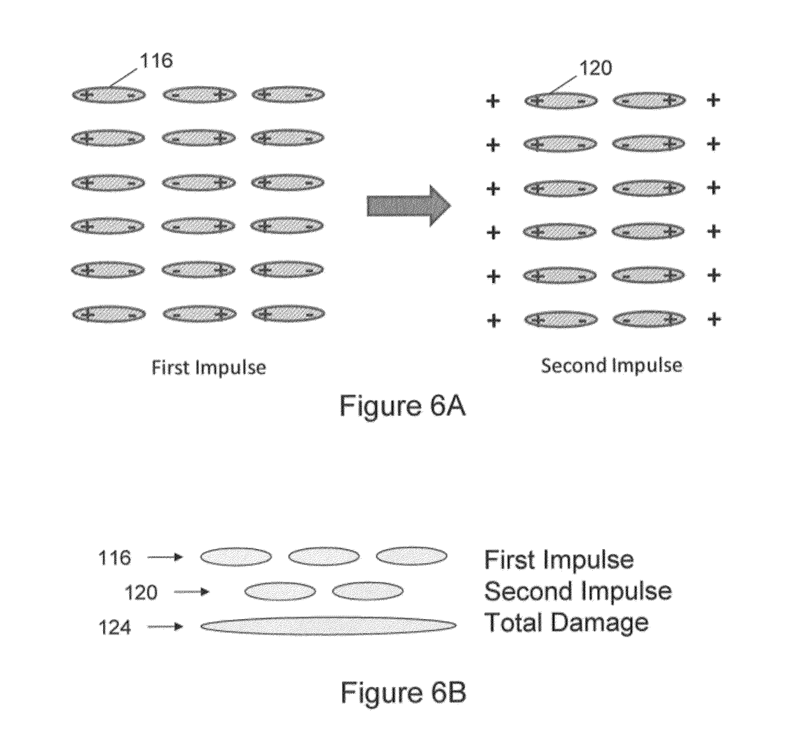

FIG. 6A shows an exemplary damage pattern for another an apparatus that can change the polarity of the needles between pulses of energy.

FIG. 6B illustrates the damage pattern formed using the technique shown in FIG. 6A.

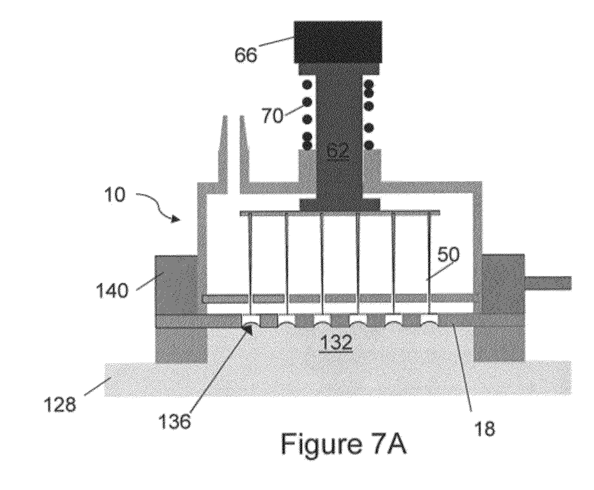

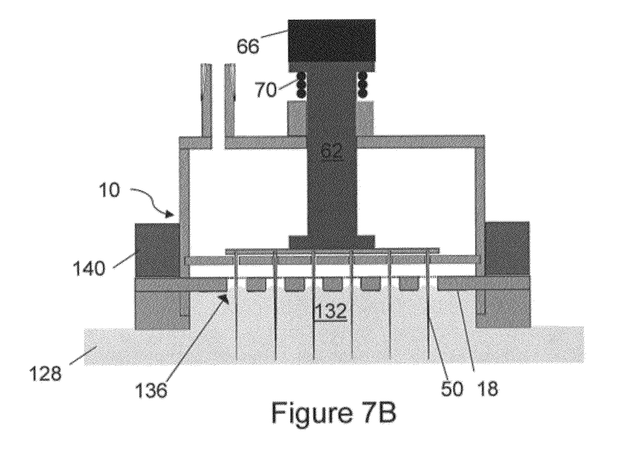

FIG. 7A shows the apparatus of FIG. 1 prior to deploying the needle array into skin.

FIG. 7B shows the apparatus of FIG. 1 with the needle array deployed into skin.

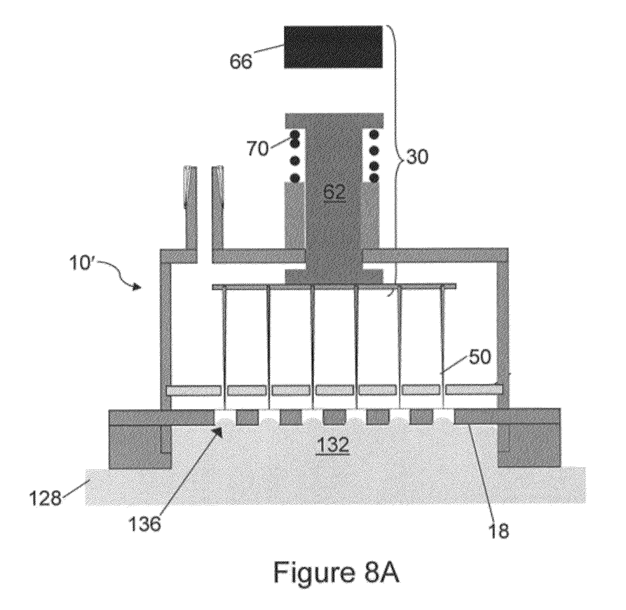

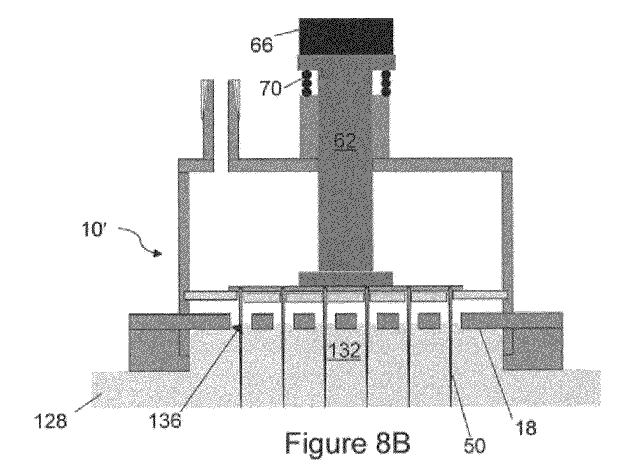

FIG. 8A shows the apparatus of FIG. 2 prior to deploying the needle array into skin.

FIG. 8B shows the apparatus of FIG. 2 with the needle array deployed into skin.

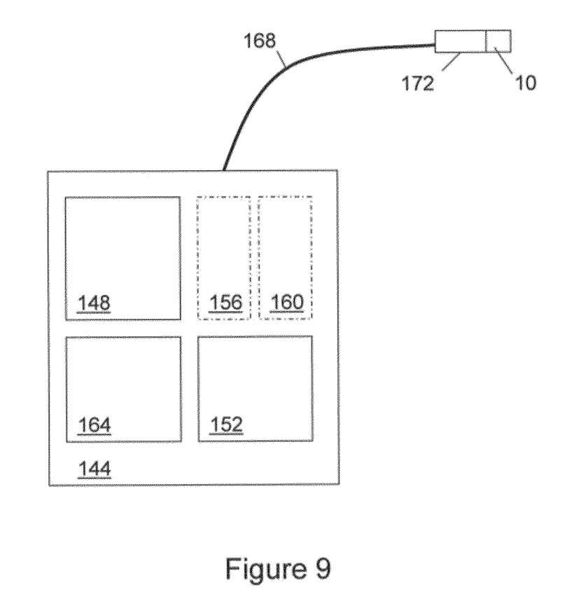

FIG. 9 shows a base station for controlling an apparatus for deploying a needle array into skin.

### DESCRIPTION OF THE INVENTION

FIGS. 1 and 2 show apparatuses 10 and 10′, respectively, for deploying a needle array into skin. Apparatuses 10 and 10′ can be referred to as cartridges. Each apparatus includes a housing 14, a plate 18 disposed in the housing 14, a vacuum port 22, a needle array 26, an actuator mechanism 30. The needle array 26 is coupled to a source of RF energy. The apparatus can optionally include a needle guide 34.

The housing 14 defines a volume 38. The housing 14 includes a ceiling 14a and a wall 14b extending from the ceiling 14a. The wall 14b has an edge 14c for contacting the skin, and the edge 14c of the wall defines an aperture 42 in the housing 14. As illustrated in FIGS. 1 and 2, the ceiling 14a defines the vacuum port 22, which can be in fluid communication with a vacuum source. In certain embodiments, the wall 14b defines the vacuum port 22.

The plate 18 intersects the wall 14b and divides the volume 38 of the housing into two sub-volumes so that the wall 14b extends above and below the plate 18. A first sub-volume 38a is defined by the ceiling 14a, the wall 14b and a top surface of the plate 18. A second sub-volume 38b is defined by a bottom surface of the plate 18, the wall 14b and the aperture 42 defined by the edge 14c of the wall 14b.

The plate 18 defines a plurality of holes 46 so that the first sub-volume 38a and the second sub-volume 38b are in fluid communication. Vacuum is applied to draw skin though the aperture 42 and into contact with the bottom surface of the plate 18. The needle array 26 includes a plurality of needles 50 extending from a base 54. Each needle 50 is aligned with a respective hole 46 defined in the plate 18.

The actuator mechanism 30 is in communication with the needle array 26. The actuator mechanism 30 is configured to cause the plurality of needles 50 to pass through their respective holes 46 into skin. A source of RF energy, radiation or current is in electrical communication with the plurality of needles 50.

In FIGS. 1 and 2, the housing 14 defines a passage 58 through the ceiling 14a. The actuator mechanism 30 includes a plunger slidably 62 insertable into the passage 58 and in communication with (e.g., connected to, removable from or attached permanently to) a top surface of the base 54. The actuator mechanism 30 is configured to slide the plunger 62 through the passage 58 to drive the plurality of needles 50 into skin. The actuator mechanism 30 can include an actuator 66 (e.g., a solenoid or a pneumatic driver) to actuate the plunger 62 and the needles 50. A spring 70 can be positioned around the shaft of the plunger 62. The spring can be positioned between a cap on the plunger 62 and a gauge stop on the housing 14. The spring 70 can retract the needle array from the skin and into the housing 14 when the actuator 66 is not powered.

In FIG. 2, the actuator 66 includes a hammer configured to strike the plunger 62 and drive the plurality of needles 50 into skin. The actuator mechanism 30 can include an actuator 66 (e.g., a solenoid or a pneumatic driver) to actuate the hammer. In this configuration, the actuator 66 can have a running period during which the actuator 66 can achieve the desired velocity for driving the needles 50 into skin. The spring 70 can retract the needle array from the skin and into the housing 14 when the actuator 66 is not powered.

The housing 14 can be formed from plastic, a polymer compound, metal, glass or sapphire. The wall can be a single continuous wall that is circular or oval. The wall can include three or more contiguous sections so that the perimeter forms a polygon such as a triangle, square, rectangle, pentagon, hexagon or similar structure.

The plate 18 can be formed from plastic, a polymer compound, metal, glass or sapphire. In various embodiments, the plate is formed from a thermally conductive material, such as metal, glass or sapphire. A cooling mechanism can be coupled to the plate so that the skin can be cooled during a treatment.

The needle array 26 can be linear, a two-dimensional lattice, circular, oval, or polygonal (e.g., hexagonal). Each needle 50 of the needle array can be identical, or the array can be composed of two or more subsets of needles. The needles can be arranged in a 6×6 array. However, any n×n square or n×m rectangular array of needles can be used (where n>1 and m>=1). The density of needles can be about 1 to about 100 per centimeter.

Each needle 50 can be formed from a metal, a ceramic, a dielectric, plastic, a polymer or a composite. In various embodiments, a needle is a medical grade acupuncture needle or a microsurgical needle. In various embodiments, a needle can be surgical grade stainless steel. A needle can be solid or can be hollow. For example, a needle can be a hypodermic needle.

A needle 50 can have a circular, oval or polygonal cross-section. The cross-sectional dimension (e.g., diameter or width) can be about 10 nm to about 1 mm. In certain embodiments, the dimension is about 1 micrometer to about 500 micrometers. In one detailed embodiment, a needle has a diameter of about 200 micrometers.

A needle 50 can be about 1 micrometer to about 5 cm in length. In certain embodiments, the length is about 500 micrometers to about 5 mm. In one detailed embodiment, the length is about 2 mm.

FIG. 3 shows an embodiment of a hollow needle 50′, which can be used to accommodate a sensor, delivery of a fluid or removal of tissue. A hollow needle 50′ can include a wall 80 that defines a bore 84. A temperature sensor 88 can be insertable into the bore 84. The temperature sensor 88 can include or be attached to a lead 92 that provides a signal to a controller. The temperature sensor 88 can be a thermistor, a thermocouple or a thermally sensitive diode, which is placed at or near the tip of the needle. Temperature can be monitored during a treatment.

A hollow needle 50′ can be used to accommodate a current sensor, a voltage sensor or an impedance sensor.

A hollow needle 50′ can also be used to introduce a fluid, such as conductive electrolytic fluid, a medication or an anesthetic. A highly conductive electrolytic fluid can form a bleb or cavity that effectively increases the surface area of the needle in contact with tissue, which can result in a larger damage zone.

FIG. 4 shows a needle 50″ having a shaft covered in an insulating material 96 and having an exposed tip 100 to deliver the RF energy. The insulating material 96 can be a polymer coating such as, for example, Teflon (polytetrafluoroethylene) or Kapton (polyimide). The insulating material 96 can insulate the epidermis and/or dermis from direct exposure to RF energy transmitted by the shaft of the needle. In one embodiment, the insulating material 96 covers 1 mm of a 2 mm needle.

A tip of a needle 50 can be flat, angled, or beveled. A sharp edge can allow effective cutting through tissue with minimal needle penetration force, which can reduce discomfort and minimal stress on a needle, which reduces the possibility of breaking a needle. A sharp point can have bevels at opposing angles to prevent hooking at the needle tip. A sharp point can have bevels at opposing angles to form three shape lines that create a “Y” shaped cut through tissue reducing tissue stretching, needle penetration force, and subject discomfort. A bevel can be used to prevent coring of the tissue where a core of tissue is cut by the needle and becomes dislodged in the lumen of the needle. A needle can form an entry wound that effectively closes when the needle is removed to prevent seepage of extracellular fluid, plasma or blood.

The base 54 can be formed from a metal, a semiconductor, a ceramic, plastic, a polymer or a composite. In certain embodiments, the base is a circuit board. Each needle can be individually soldered to the circuit board.

The actuator mechanism 30 can include an electric solenoid or a pneumatic plunger. The actuator mechanism 30 can be used to rapidly insert needles into tissue and facilitate penetration. A “push-type,” intermittent duty 12-volt DC solenoid can be used as it offers higher pushing powers compared to continuous duty solenoids. The solenoid can be mechanically coupled to a plunger attached to the needle plate. The actuator mechanism 30 or the actuator 66 can be tuned to drive the needles at a velocity greater than or about 100 mm/s. The velocity can be greater than about 1 m/s.

The needle guide 34 can include close fitting guide holes for each needle. The needle guide 34 can be used to prevent bending or buckling of the needles as the needles are rapidly inserted into skin or removed from the skin. The guide plate holes can be formed with a nonconductive material and designed to have little friction with the needles. The guide plate holes can be lined with a nonconductive material and designed to have little friction with the needles. For example, the guide plate holes can be lined with or made from Teflon.

The depth of needle insertion is defined by a needle depth gauge stop, which can be the plate 18, the needle guide 34 or a portion of the housing 14 that catches the plunger 62. The actuator continues to advance until the base contacts the plate 18 or the needle guide 34, or until a cap on the plunger 62 comes in contact with the gauge stop on the housing 14.

In various embodiments, the needles can be selected to deliver the RF energy to a predetermined depth. In some embodiments, the RF energy is delivered to the target region about 0.005 mm to about 10 mm below the surface of the skin, although shallower or deeper depths can be selected depending on the application. In some embodiments, the depth is about 0.5 mm to about 5 mm.

A variety of needle cartridges can be supplied calibrated to set insertion depths. Alternatively, the gauge stop can be manufactured such that the insertion depth can be adjusted to set levels by rotating the plunger shaft or some other equivalent means. A compression spring can be positioned around the shaft of the plunger to retract the needle array into the cartridge when the actuator is not powered.

Vacuum suction can be used to facilitate needle insertion in three ways. First, vacuum suction can pull skin up and compress it onto a hard surface which can reduce pain. The faster firing myelinated pressure receptors effectively block signals from the slower non-myelinated pain receptors forcing the brain to only feel the sensation of pressure. This mechanism is commonly referred to as the gate theory of pain control. In general, absolute vacuum pressures between 200 mmHg and 700 mmHg is adequate to compress and hold skin, and reduce pain without causing adverse effects to skin.

Second, by compressing skin onto a hard surface, vacuum suction can assist in controlling the depth of insertion by preventing skin displacement from the surface by the needles leading to random uncontrollable needle insertion depth.

Third, vacuum suction can pull skin up into the needle holes of the plate to form a bleb of skin in each hole. This can reduce the elasticity of skin, aid needle insertion and prevent tissue displacement by the needles. In effect, the stretching force is localized to the immediate area around the needle tip insertion point, instead of spreading the force across the entire area being treated.

To avoid continuous running of the vacuum pump at max power, which is inefficient and can be noisy, the pump can be designed to idle at low power until contact is made at the skin surface. The system can ramp to high power after being triggered by a switch sensor, a drop in pressure upon tissue contact or as the edge of the housing is placed near or adjacent the skin surface. The vacuum pump can pump the housing through a small orifice when the housing is not in contact with the skin. When the housing is brought into contact with the skin, a drop in the pressure in the housing can be sensed to open a valve which bypasses the orifice and allow full flow of air into the pump to allow the pressure in the housing to be lowered quickly.

In addition to serving as a hard flat plate for vacuum suction, the perforated needle plate can cool superficial skin layers (e.g., epidermis and/or dermis) to avoid thermal injury to these layers. Thermal models show that rapid cooling of the epidermis and papillary dermis is possible (e.g., a temperature decrease of about 20° C. in less than 0.5 seconds). By numbing the more superficial pain and pressure nerves, skin cooling can aid patient comfort. Cooling can be effected be running a cooling fluid (cryogen, water, a cooling gas, etc.) across a portion of the plate or through the plate, or by attaching a Stirling cooling or other cooling device to the plate.

The system can be designed to deliver monopolar RF energy or bipolar RF energy. In a bipolar mode, biaxial alternating poles can be used. For example, every alternating needle can be connected to the first RF pole and the remaining alternative needles can be connected to the second RF pole. All needles can be powered by a single bipolar connection, although each pair of needles can be connected separately to enable precise control of current through individual needle pairs.

FIG. 5 shows an example of a damage pattern 104 for biaxial alternating poles. Zones 108 are formed by a first subset of needles having a first polarity, and zones 112 are formed by a second subset of needles having an opposite polarity (e.g., 180 degrees out of phase with the first polarity). The thermal damage pattern can be constrained around the needle tip. This fractional damage pattern can be similar to a pattern formed by monopole needles.

FIG. 6A shows an exemplary damage pattern for an apparatus that can change the polarity at the poles between pulses of energy. The polarity of the poles along a single axis can be arranged to form a first subset 116 of spaced injuries during a first pulse, and the polarity of the poles along the same axis can be changed to form a second subset 120 of spaced injuries during a second pulse. As shown in FIG. 6B, the second subset 120 can be caused between the first subset 116, so that a linear ribbon 124 of damage along a single axis is formed. In this design, the polarity of the poles is selected to that the current flows substantially between pairs of electrodes, and damage is formed between the electrode pairs. Lower power, longer impulses can prevent coagulating damage around the needle tips and prevent substantial damage between the electrodes. By shifting the polarity of the poles along one axis, the area of damage is extended to a continuous ribbon along this axis. This approach can be used for treating deep wrinkles where tightening skin on a single axis perpendicular to the wrinkles is preferred.

FIG. 7A shows apparatus 10 placed in contact with skin 128. Vacuum has been applied to draw a portion 132 of the skin into the aperture 42 and against the plate 18. The surface of the skin can be drawn flat against the plate, e.g., to ensure uniform insertion depth of needles. Blebs 136 of skin form in the holes 46 of the plate 18, which can aid needle insertion. The spring 70 is in an expanded state. FIG. 7B shows apparatus 10 with its needles 50 driven into the skin 128 by the actuator mechanism 30. The spring 70 is in a compressed state, and can be used to withdraw the needles 50 from the skin 128.

FIGS. 7A and 7B also show an embodiment of a cooling mechanism 140 coupled to the plate 18. In this illustrative embodiment, the cooling mechanism 140 is a manifold that circulates a cooling fluid such as water across a portion of the plate 18 to extract heat from the plate 18. The cooling fluid can be contained within the cooling mechanism cooling the plate by thermal conduction. Alternatively, the cooling fluid can be passed through the plate to directly cool the plate.

FIG. 8A shows apparatus 10′ placed in contact with skin 128. Vacuum has been applied to draw a portion 132 of the skin into the aperture 42 and against the plate 18. Blebs 136 of skin are formed in the holes 46 of the plate 18. The actuator 66 is spaced from the plunger 62. The spring 70 is in an expanded state. FIG. 8B shows that actuator 66 has struck the plunger 62 and driven the needles 50 into the skin 128. The spring 70 is in a compressed state, and can be used to withdraw the needles 50 from the skin 128. The cooling mechanism 140 shown in FIG. 7 can be used with apparatus 10′.

FIG. 9 shows an exemplary base station 144 for controlling an apparatus 10 or 10′ for deploying a needle array 50 into skin. The base station 144 houses an RF source 148, a controller 152, a vacuum source 156, a reservoir 160, and a power source 164. The base station 144 is connected by an umbilicus 168 to a handpiece 172 including the RF needle assembly 10 or 10′.

Controller 152 can allow the user to set the RF power and adjust the RF exposure time to coincide with a desired amount of energy delivered to tissue. The controller 152 can control the timing and sequence of steps required for ideal treatment. The controller 152 can control one or more of the following steps:

1. pulling vacuum on skin and pressing skin against the plate,

2. a set delay allowing for skin cooling,

3. quickly driving the needles into skin,

4. delivering the RF energy,

5. retracting the needles,

6. a set delay to allow for additional post-RF cooling, and

7. turning off vacuum.

The treatment sequence can be triggered by pressing a switch on the handpiece or by stepping on a footswitch. The sequence can be terminated prematurely by releasing the switch. Otherwise, termination can occur automatically based on the timing of the sequence or if a preset maximum time is reached.

The rapid driving of needles can reduce the insertion force on the skin thereby ensuring complete needle insertion of all needles without pushing the skin away from the plate. Vacuum suction can compress tissue against a flat plate to ensure needle insertion to a desired and controlled depth. Vacuum suction can compress tissue against a flat plate to reduce pain (e.g., a gate theory effect). The use of surface cooling can reduce pain and guarantee no heating injury to the epidermis and papillary dermis by the conducted heat from the RF heated tissue underneath. Insulation on the shaft of the needles can protect the epidermis or dermis from injury from the RF energy. Insulation also prevents electrical shorts across electrically conductive cooling plates.

Controller 152 can be used to automate the treatment sequence, which includes vacuum application, time delay for surface cooling, needle insertion, RF power application, needle withdrawal, time delay for post cooling, and finally vacuum suction release. Automation not only makes the system easy to use, but also ensures RF energy delivery in a repeatable manner.

The front panel interface of the base station 144 can be designed to allow the user to adjust RF parameters and time delays. User inputs include, but are not limited to, RF power, delivered RF energy, time delay to hold vacuum and pre-cool the skin surface, time delay for post-cooling, maximum time for RF exposure, and maximum time for vacuum suction at high power.

The RF exposure time can be pre-set or can be adjusted real-time by monitoring the energy delivered to tissue. The energy delivered to tissue can be monitored in total or individually to each needle. The amount of damage formed around each needle tip can be constant and reproducible from treatment site to treatment site. This is particularly important to account for site to site variations in tissue impedance and for impedance changes that occur during RF exposure. The RF treatment time can be pre-set or adjusted in real-time by monitoring the temperature at each needle site. The treatment can be stopped when a desired temperature is reached. As a safety feature, the front panel interface can allow setting a maximum RF exposure time where RF energy is turned off once the desire energy is delivered or the maximum exposure time has been reached.

The following is an exemplary list of operating specifications for a microneedle array for treating skin with RF radiation. The parameters can be used to shrink dermal collagen. The numbers are intended to show values for best use as currently understood. Variations of these values are possible.

| | |
|---|---|
| RF Frequency 460 kHz | (400 kHz to 10 MHz) |
| RF Power (for 36 needle array) | 30 W (0.3 W to 50 W) |
| RF Energy Delivered | 15 J (10 J to 30 J) |
| (for 36 needle array) | |
| Needle diameter | 0.2 mm |
| Needle array and spacing | 6 × 6, 2.2 mm center to center |
| Needle penetration depth | 2.0 mm (0.5 mm to 5 mm) |
| Exposed distal needle tip length | 0.5 mm |
| Vacuum suction | 200 to 700 mmHg absolute pressure |
| Contact cooling | 0-10° C. (ideally about 5° C.) |

The range for RF power and energy scales with the number of needles used. Ranges are typical for a 6×6 needle array. A rough estimate of the thermal damage volume around each needle can be calculated from the following equation:

$$Vol = \frac{0.33 J / needle}{( 1.2 g / {cm}^{3} ) \times ( 3.3 J / g - ° C ) \times 35 ° C} = 0.0024 {cm}^{3} / needle .$$

This corresponds to roughly a 1.5 mm diameter spherical damage zone.

The reservoir 160 can be used to house the fluid delivered by a hollow needle. The reservoir 160 also can be used to house the cooling fluid used by the cooling mechanism 140 for the plate 18.

The umbilicus 168 can include conduits for power, RF energy, control signals, and fluids being delivered from the base station 144 to the handpiece 172.

Apparatus 10 or 10′ can be a portion of handpiece 172, or can be attachable to and/or removable from handpiece 172. All or a portion of the actuator mechanism 30 can be contained within the handpiece 172. For example, the actuator 66 can be included in or a portion of the handpiece 172. The plunger 62 can retract into the handpiece 172.

The apparatus 10 or 10′ can be a single use disposable needle cartridge that can snap into or onto handpiece 172. The cartridge can include suitable electrical connections so that electrical current and/or RF power can be delivered from the base station 144 to the handpiece 172 to the cartridge, and the cartridge can include suitable connectors to receive fluids or vacuum from the base station 144. For example, the electrical connections can couple RF energy to the needle plate, a vacuum line and connection can deliver vacuum suction to the needle cartridge, a cooling line and return can deliver chilled coolant to a cooling manifold, and a trigger line can activate the treatment sequence.

In some embodiments, the RF energy can be delivered to the adipose tissue layer to thermally injure, damage, and/or destroy one or more fat cells. The RF energy can damage one or more fat cells so that at least a portion of lipid contained within can escape or be drained from the treated region. At least a portion of the lipid can be carried away from the tissue through biological processes. In one embodiment, the body's lymphatic system can drain the treated fatty tissue from the treated region. In an embodiment where a fat cell is damaged, the fat cell can be viable after treatment. In one embodiment, the treatment radiation can destroy one or more fat cells. In one embodiment, a first portion of the fat cells is damaged and a second portion is destroyed. In one embodiment, a portion of the fat cells can be removed to selectively change the shape of the body region.

In some embodiments, RF heating can occur in selective targets within or outside the tissue layer being targeted. For example, suitable targets for RF heating include, but are not limited to, a fat cell, lipid contained within a fat cell, fatty tissue, a wall of a fat cell, water in a fat cell, and water in tissue surrounding a fat cell. The energy absorbed by the target can be transferred to nearby fat cell to damage or destroy the fat cell. For example, thermal energy absorbed by dermal tissue can be transferred to the fatty tissue. In one embodiment, the RF energy is delivered to water within or in the vicinity of a fat cell in the target region to thermally injure the fat cell. For example, dermal heating can be transferred to sebaceous glands to clear acne.

In various embodiments, treatment radiation can cause sufficient thermal injury in the dermal region of the skin to elicit a healing response to cause the skin to remodel itself. This can be done alone, or in conjunction with a treatment that affects one or more fat cells. This can result in more youthful looking skin and an improvement in the appearance of cellulite. In one embodiment, sufficient thermal injury induces fibrosis of the dermal layer, fibrosis on a subcutaneous fat region, or fibrosis in or proximate to the dermal interface. In one embodiment, the treatment radiation can partially denature collagen fibers in the target region. Partially denaturing collagen in the dermis can induce and/or accelerate collagen synthesis by fibroblasts. For example, causing selective thermal injury to the dermis can activate fibroblasts, which can deposit increased amounts of extracellular matrix constituents (e.g., collagen and glycosaminoglycans) that can, at least partially, rejuvenate the skin. The thermal injury caused by the radiation can be mild and only sufficient to elicit a healing response and cause the fibroblasts to produce new collagen. Excessive denaturation of collagen in the dermis causes prolonged edema, erythema, and potentially scarring. Inducing collagen formation in the target region can change and/or improve the appearance of the skin of the target region, as well as thicken the skin, tighten the skin, improve skin laxity, and/or reduce discoloration of the skin.

In various embodiments, a zone of thermal injury can be formed at or proximate to the dermal interface. Fatty tissue has a specific heat that is lower than that of surrounding tissue (fatty tissue, so as the target region of skin is irradiated, the temperature of the fatty tissue exceeds the temperature of overlying and/or surrounding dermal or epidermal tissue. For example, the fatty tissue has a volumetric specific heat of about 1.8 J/cm³ K, whereas water in skin has a volumetric specific heat of about 4.3 J/cm³ K. In one embodiment, the peak temperature of the tissue can be caused to form at or proximate to the dermal interface. For example, a predetermined needle depth, RF energy, and cooling parameters can be selected to position the peak of the zone of thermal injury at or proximate to the dermal interface. This can result in collagen being formed at the bottom of the dermis and/or fibrosis at or proximate to the dermal interface. As a result, the dermal interface can be strengthened against fat herniation into the dermis, thereby improving the appearance of cellulite. For example, strengthening the dermis can result in long-term improvement of the appearance of the skin since new fat being formed or untreated fat proximate the dermal interface can be prevented and/or precluded from crossing the dermal interface into the dermis.

In one embodiment, fatty tissue is heated by RF energy, and heat can be conducted into dermal tissue proximate the fatty tissue. The fatty tissue can be disposed in the dermal tissue and/or can be disposed proximate to the dermal interface. A portion of the dermal tissue (e.g., collagen) can be partially denatured or can suffer another form of thermal injury, and the dermal tissue can be thickened and/or be strengthened as a result of the resulting healing process.

In one embodiment, water in the dermal tissue is heated by the RF energy. The dermal tissue can have disposed therein fatty tissue and/or can be overlying fatty tissue. A portion of the dermal tissue (e.g., collagen) can be partially denatured or can suffer another form of thermal injury, and the dermal tissue can be thickened and/or be strengthened as a result of the resulting healing process. A portion of the heat can be transferred to the fatty tissue, which can be affected. In one embodiment, water in the fatty tissue absorbs radiation directly and the tissue is affected by heat.

In various embodiments, a treatment can cause minimal cosmetic disturbance so that a patient can return to normal activity following a treatment. For example, a treatment can be performed without causing discernable side effects such as bruising, open wounds, burning, scarring, or swelling. Furthermore, because visible side effects are minimal, a patient can return to normal activity immediately after a treatment or within a matter of hours, if so desired.

A processor or control module suitable for the execution of computer programs include, by way of example, both general and special purpose microprocessors, and any one or more processors of any kind of digital computer. A processor can receive instructions and data from a read-only memory or a random access memory or both. A processor also includes, or be operatively coupled to receive data from or transfer data to, or both, one or more mass storage devices for storing data, e.g., magnetic, magneto-optical disks, or optical disks. Data transmission and instructions can also occur over a communications network.

Information carriers suitable for embodying computer program instructions and data include all forms of non-volatile memory, including by way of example semiconductor memory devices, e.g., EPROM, EEPROM, and flash memory devices; magnetic disks, e.g., internal hard disks or removable disks; magneto-optical disks; and CD-ROM and DVD-ROM disks. The processor and the memory can be supplemented by, or incorporated in special purpose logic circuitry.

While the invention has been particularly shown and described with reference to specific embodiments, it should be understood by those skilled in the art that various changes in form and detail may be made therein without departing from the spirit and scope of the invention as defined by the appended claims.

What is claimed:

1. An apparatus for treating skin, comprising:
a housing defining a volume, the housing including a ceiling and a wall extending from the ceiling, the wall having an edge for contacting the skin, the edge of the wall defining an aperture in the housing;
a plate disposed in the housing, the plate intersecting the wall and dividing the volume of the housing into two sub-volumes so that the wall extends above and below the plate, wherein (i) a first sub-volume is defined by the ceiling, the wall and a top surface of the plate, (ii) a second sub-volume is defined by a bottom surface of the plate, the wall and the aperture defined by the edge of the wall, and (iii) the plate defines a plurality of holes so that the first sub-volume and the second sub-volume are in fluid communication;
a vacuum port defined in the ceiling or the wall of the housing, vacuum applied to draw the skin though the aperture and into contact with the bottom surface of the plate;
a needle array comprising a plurality of needles extending from a base, each needle aligned with a respective hole defined in the plate;
an actuator mechanism in communication with the needle array, the actuator mechanism configured to cause the plurality of needles to pass through their respective holes into the skin; and
a source of RF energy in electrical communication with the plurality of needles.

2. The apparatus of claim 1 wherein each needle includes (i) a shaft covered with an insulating material and (ii) an exposed tip to deliver the RF energy.

3. The apparatus of claim 1 wherein the actuator mechanism comprises a solenoid.

4. The apparatus of claim 1 wherein the actuator mechanism is adapted to drive the plurality of needles into the skin at a velocity of at least 100 mm/s.

5. The apparatus of claim 1 wherein the actuator mechanism is adapted to drive the plurality of needles into the skin at a velocity of at least 1 m/s.

6. The apparatus of claim 1 wherein (i) the housing defines a passage through the ceiling and (ii) the actuator mechanism comprises a plunger slidably insertable into the passage and connected to a top surface of the base, the actuator mechanism configured to slide the plunger through the passage to drive the plurality of needles into the skin.

7. The apparatus of claim 1 wherein the actuator mechanism comprises a hammer configured to strike the base of the plurality of needles and drive the plurality of needles into the skin.

8. The apparatus of claim 1 wherein at least one needle is hollow.

9. The apparatus of claim 8 further comprising a temperature sensor inserted into a bore defined in the at least one needle.

10. The apparatus of claim 1 wherein a first subset of the plurality of needles has an oscillating polarity and a second subset of the plurality of needles has a polarity opposite of the first.

11. The apparatus of claim 1 further comprising a cooling mechanism for the plate, wherein the plate is thermally conductive.

12. A method of treating skin, comprising:
applying vacuum to draw skin into contact with a plate defining a plurality of holes;
inserting a plurality of needles through the plurality of holes into the skin; and
delivering RF energy via the plurality of needles to treat the skin.

13. The method of claim 12 further comprising inserting the plurality of needles into the skin at a velocity of at least 100 mm/s.

14. The method of claim 12 further comprising inserting the plurality of needles into the skin at a velocity of at least 1 m/s.

15. The method of claim 12 further comprising applying the vacuum to form a bleb of skin in each hole.

16. The method of claim 12 wherein the plate is disposed in a housing defining a volume, the housing including a ceiling and a wall extending from the ceiling, the wall having an edge for contacting the skin, the edge of the wall defining an aperture in the housing, the plate intersecting the wall and dividing the volume of the housing into two sub-volumes so that the wall extends above and below the plate, wherein (i) a first sub-volume is defined by the ceiling, the wall and a top surface of the plate, (ii) a second sub-volume is defined by a bottom surface of the plate, the wall and the aperture defined by the edge of the wall, and (iii) the first sub-volume and the second sub-volume in fluid communication via the plurality of holes.

17. The method of claim 12 further comprising cooling the plate.

18. A method of treating skin, comprising:
applying vacuum to draw skin into contact with a plate defining a plurality of holes, the plate disposed in a housing defining a volume, the housing including a ceiling and a wall extending from the ceiling, the wall having an edge for contacting the skin, the edge of the wall defining an aperture in the housing, the plate intersecting the wall and dividing the volume of the housing into two sub-volumes so that the wall extends above and below the plate, wherein (i) a first sub-volume is defined by the ceiling, the wall and a top surface of the plate, (ii) a second sub-volume is defined by a bottom surface of the plate, the wall and the aperture defined by the edge of the wall, and (iii) the first sub-volume and the second sub-volume in fluid communication via the plurality of holes inserting into the skin a plurality of needles through the plurality of holes at a velocity of at least 100 mm/s; and
delivering RF energy via the plurality of needles to treat the skin.

19. The method of claim 18 further comprising applying the vacuum to form a bleb of skin in each hole.
